SEQUENCE LISTING (2 sequences)

SEQ ID NO: 1 - protein, 214 aa, Carica candamarcensis
YPESIDWRQKGAVTPVKDQNPCGSCWAFSTVATVEGINKIVTGKLISLSEQELLDCDRRSHGCKGGYQTTSLQYVVDNGVHTEKVYPYEKKQGKCRAKDKQGPWVKITGYKRVPSNDEISLIKAIATQPVSVLVESKGRAFQFYKGGVFGGPCGTKLDHAVTAVGYGKDYILIKNSWGLRWGDKGYIKIKNASGNSEGICGVYKSSYFPIKGYQ

SEQ ID NO: 2 - protein, 214 aa, Carica candamarcensis
YPGSVDWRZKGAVTPVGDQNPCGSCWAFSTVATVEGINKIVTGHLISLSEQELLDCDRRSHGCKGGYQTGSLQYVVDYGVHTEYVYPYEKKQGKCRAKDKQGPKVQITGYKRVPTNDEISLIKVIANQPVSKLIESKGRSFHFYRGGIYKGPCGTRLDHAVTAIGYGKDYILIKNSWGPNWGEKGYIKIKNASGKSEGICGVYKSSYFPTKEYQ

(19) United States
(12) Patent Application Publication
(10) Pub. No.: US 2009/0005303 A1
(43) Pub. Date: Jan. 1, 2009

(54) PROTEASES FROM CARICA HAVING MITOGENIC ACTIVITY AND THEIR METHODS OF USE

(75) Inventors: Carlos E. Salas, Belle Horizonte (BR); Ruben Dario Sinisterra Millan, Bello Horizonte (BR); Miriam T. P. Lopes, Bello Horizonte (BR)

Correspondence Address: ROSENBAUM & ASSOCIATES, P.C., 650 DUNDEE ROAD, SUITE #380, NORTHBROOK, IL, 60062, US

(73) Assignee: UNIVERSIDADE FEDERAL DE MINAS GERAIS-UFMG (Federal University of Minas Gerais), Bello Horizonte (BR)

(21) Appl. No.: 11/778,488

(22) Filed: Jul. 16, 2007

Related U.S. Application Data
(63) Continuation-in-part of application No. 11/378,196, filed on Mar. 17, 2006, now Pat. No. 7,258,856.
Application No. 11/378,196 is a continuation-in-part of application No. 10/162,267, filed on Jun. 3, 2002, now abandoned.

(51) Int. Cl. A61K 38/16; A61P 17/02

(52) U.S. Cl. 514/12

(57) ABSTRACT

Proteases having mitogenic activity isolated from the genus Carica are provided. In particular the proteases are cysteine proteases isolated from Carica candamarcensis. In addition, the recombinant forms of the protease, including fragments and mutants with substantial homology are provided. Also provided are pharmaceutical compositions useful for treating wounds that include the disclosed proteases with mitogenic activity. A method of treating wounds is provided using the disclosed proteases.

### REFERENCE TO CROSS RELATED APPLICATIONS

The present application is a continuation-in-part of application U.S. patent application Ser. No. 11/378,196, filed Jul. 13, 2006, which is a continuation-in-part of application U.S. patent application Ser. No. 10/162,267, filed Jun. 3, 2002, the disclosure of which are incorporated herein by reference.

### FIELD OF INVENTION

The present invention relates to a group of proteolytic enzymes or proteases isolated from the genus Carica. In particular, the proteolytic enzymes are cysteine proteases that function as mitogenic stimulators of mammalian cells. The present invention also relates to a process for the production of these enzymes and their use as a wound healing promoter.

### BACKGROUND

The skin is an important organ for homeostasis and host defense against foreign invaders. Specifically, it acts as the body's first line of defense against infection. Accordingly, it is important that lesions or wounds in the skin be rapidly closed to prevent infection. Some types of wounds, however, are resistant to healing under normal physiological conditions.

The process of wound healing involves a complex system of local and remote (systemic) resources. For example, amino acids and sugars are needed as substrates for collagen and proteoglycan synthesis. Migration of fibroblasts and epithelial/endothelial cells during the wound healing process places additional systemic demands on a subject during the wound healing process. Wounded tissues have unique nutritional needs and physiological features. Lymphocyte participation in wound healing has been demonstrated. Alteration in the hosts T-cell dependent immune response has also been shown to influence wound healing. Cyclosporine and anti T-cell antibodies, both of which interfere with T-cell function, abrogate wound healing. Similarly, macrophages and their products are also involved in wound healing. Increased circulation usually results in rapid delivery of monocytes and PMN's to the wound site. This in turn results in the elimination of bacterial contamination of the wound due to nonspecific killing mechanisms and also enhances the rate of wound healing. These various cell types are synthesized by the bone marrow.

While wound healing is typically an efficient and natural process that normally requires no special treatment, chronic non-healing wounds can occur. In the chronic cases, there is some underlying factor preventing healing and intervention is often necessary to complete the healing process. For example, pressure sores are initially acute wounds caused by ischemic death of tissue due to excessive pressure and will usually heal readily when pressure is relieved and the blood supply restored. Often times it is difficult to resolve these causative factors and chronic wounds can develop. Most of these chronic wounds are characterized by the accumulation of devitalized tissue and cellular exudates at the outer surface. These products result from a restriction of nutrients to the damaged epithelium and form either a dry, hard eschar or, as in the case of deep moist wounds, a slough that frequently hardens on the outside with exposure to the air. The accumulation of these products in the wound bed is generally regarded to prevent or delay granulation and epithelialisation. The removal of this tissue by a process termed debridement is therefore thought to facilitate healing.

Debridement can be accomplished by both mechanical and non-mechanical methods. The mechanical methods require the physical elimination of the devitalized tissue from the healthy, but this difficult and often results in the aggravation of the wound. There are various non-mechanical debridement methods that include enzymes, hydrogels and chemical formulations. While various methods of debridement exist, there is no proven reliability of any particular method of debridement with respect to a particular wound. In particular, use of proteolytic enzymes in the early debridement (digestion and separation) of eschar tissues, such as in burn wounds, decubitus ulcers, pressure necroses and bed sores has been researched, e.g., streptokinase, trypsin and papain.

There remains a need for isolating and providing an agent that acts as an effective promoter of wound healing.

### SUMMARY OF INVENTION

Aspects of the present invention satisfy the unmet needs in the art, as disclosed above. In particular, the invention provides a natural protease isolated from Carica, or its metabolites, along with recombinant forms of the natural protease, including fragments and mutants, that exhibit protease activity comparable to the wild type protease.

Another aspect of the present invention provides a pharmaceutical composition comprising an amount of protease effect for wound treating, wherein the protease comprises one of the proteins disclosed herein, which includes the natural, recombinant, fragment, and mutant forms of the protease.

In another aspect of the present invention, a method of treating wounds is provided using the compositions disclosed herein. The compositions containing the proteins of the present invention are used for treating various wounds, including chronic wounds like ulcers.

Still another aspect of the present invention provides a protease having mitogenic or proliferative activity. The protease is preferably a cysteine protease and more preferably a protease isolated from Carica, particularly Carica candarmacensis.

These and other aspects of the invention will be obvious to those of ordinary skill in the art considering the provided disclosure and examples.

### BRIEF DESCRIPTION OF THE DRAWINGS

FIG. 1 shows the Fourier transform infrared (FTIR) spectra of P1G10: β-cyclodextrin (βCD).

FIGS. 2A and 2B show the thermogravimetric analysis (TG) and differential thermogravimetric analysis (DTG) of P1G10: β-cyclodextrin (βCD).

FIG. 3 shows the differential scanning calorimetry (DSC) of P1G10: β-cyclodextrin (βCD).

FIG. 4 shows the X-Ray powder diffraction (XRD) pattern of P1G10: β-cyclodextrin (βCD).

FIGS. 5A and 5B show 1H-nuclear magnetic resonance (NMR) of P1G10: β-cyclodextrin (βCD). The 1H-NMR profiles at 400 MHz were obtained for βCD, as shown in FIG. 2A and P1G10: βCD (1:1 w/w), as shown in FIG. 2B to assess the host:guest interaction. The dotted circles show relevant regions of chemical shift.

FIG. 6 shows Circular dichroism (CD) of P1G10: β-cyclodextrin (βCD).

FIGS. 7A, 7B, and 7C shows Isothermal titration calorimetry (ITC) of P1G10: β-cyclodextrin (βCD). FIG. 7A is the control curve after injection of P1G10 133 g/L in water MQ and titration curve of P1G10 133 g/L in βCD 5 mmol/L. FIG. 7B is the raw data of isothermal titration calorimetry. FIG. 7C is the adjusted titration curve after subtraction of control curve.

FIG. 8 shows the amidase activity of P1G10: β-cyclodextrin (βCD) complex.

### DETAILED DESCRIPTION OF PREFERRED EMBODIMENTS

The present invention relates to proteases, more particularly, cysteine proteases, and the production and use thereof. In particular, the one aspect of the present invention relates to a group of cysteine proteases, termed CC23a-e. This group of cysteine proteases is characterized as immunologically distinct from papain, having a molecular mass of about 23,000 Da and having a distinct net charge.

In another aspect of the present invention, a process for the production of the disclosed cysteine proteases is provided comprising separation of the protease from latex or leaf plant material of Caricaceae and purification therefrom, including Carica candarmacensis and Carica papaya.

The disclosed cysteine proteases can be used to enhance the process of debridement and wound healing of eschar tissue resulting from burn wounds, sores and ulcers, for example. Another aspect of the present invention comprises an enzyme preparation specifically adapted for use in enhancing the process of debridement and wound healing.

Another aspect of the present invention provides a pharmaceutical composition which comprises at least one of the present cysteine proteases CC23a-e and at least one pharmaceutically-acceptable carrier.

As indicated above, the cysteine proteases CC23a-e have a relative molecular mass of 23,000 Da and are highly basic proteins, having a pI greater than 9.5, and exhibit faster migration in a cathodal electrophoresis system. CC23a-e require thiol compounds for full activity and is inhibited by the class-specific inhibitors E-64 [L-3-carboxy-2,3-trans-epoxypropionyl-leucylamido (4-guanidino) butane] and chicken cystatin.

These cysteine proteases may be obtained by conventional preparative fast protein liquid chromatography (FPLC) cation-exchange chromatography at pH 9.2. Currently, commercially available crude latex extract powders (Technologic Farm) are commonly used as a source of the present proteases. More specifically, an extract may be chromatographed using a cation exchanger (Mono S) using a Pharmacia FPLC system and a gradient of 0.005M to 1.0M sodium chloride at pH 9.0. A variety of substrates may be used to assay the fractions for cysteine protease activity. The different proteases can be identified on the basis of their different substrate specificities.

The proteases can also be produced using recombinant DNA techniques. The sequence of a cysteine protease, e.g., CC23a, CC23b, and CC23c, is determined using known protein sequencing techniques, including Edman degradation. Based on the determined amino acid sequence, DNA primers are synthesized. These DNA primers can be used in RT-PCR reactions to select the cDNA which code for this particular cysteine protease. Once the identity is confirmed, the recombinant cDNA can be cloned into an appropriate expression vector using well-known cloning techniques. The resulting expression vector can be used to transform an appropriate cell line to express the recombinant cysteine protease, which can be isolated using standard purification techniques. The proliferative property of the recombinant cysteine protease can be tested as done with the naturally derived protease.

Preferred mutants or fragments of the cysteine proteases disclosed herein have corresponding amino acid sequences, in relation to the natural amino acid sequence, that are substantially homologous. Substantially homology is used to describe amino acid sequences that have near identity to the naturally occurring protease but contain substitutions that do not greatly alter function. Specifically, substantially homologous means a protein having a sequence that has at least about 80%, usually at least about 90% and more usually at least about 98% sequence identity with the sequence of the disclosed cysteine protease, as measured by BLAST.

Site-directed mutagenesis can be used to create mutations of the proteases that still retain proliferative activity. Preferably, conservative mutations are contemplated. The conservative substitutions can be introduced by modification of DNA encoding for the polypeptides of the invention. A conservative substitution is recognized in the art as a substitution of one amino acid for another amino acid that has similar properties, typically allowing the expression of a functional protein.

The present cysteine proteases may be admixed with pharmaceutically-acceptable carriers for application to patients. The generally preferred route of administration is by topical application to the site of a wound or ulcer. The pharmaceutical preparation may be in the form of a sterile solution that is applied to an inert dressing, such as a gauze pad, a gel, or ointment that is placed directly on the wound.

Such pharmaceutical compositions may contain the cysteine protease(s) in an active form, or, preferably, in an inactive form in which the active site cysteine residue forms half of a disulphide bridge. The other half comprises a pharmaceutically-acceptable thiol compound, for example the amino acid cysteine. The present formulations may be prepared immediately before use by mixing a lyophilised preparation of the cysteine protease with an aqueous solution. If an enzyme is present in an inactive form, an activating agent, for example cysteine, must be added to regenerate the free active site thiol of the protease.

The cysteine proteases described herein are proliferative factors useful for enhancing the healing of wounds. An amount of the protease effective for wound healing is readily determined by one of ordinary skill in the art using standard techniques, and such an amount is applied to the wound by standard techniques known in the art. Preferably, the amount of protease effective for wound healing is a concentration ranging from about 50 ng/ml to about 500 ng/ml as a single application, or in dosing regimens that range from several times per day to once every few days for a period of one to several weeks. In a topical formulation, the amount effective for wound healing is about 0.01 μg/cm² to about 100 μg/cm² of cysteine protease administered directly to the wound. These cysteine proteases can be used to treat many types of chronic non-healing wounds, such as fall-thickness dermal ulcers, e.g., pressure sores, venous ulcers, and diabetic ulcers; to treat acute wounds such as burns, incisions, and injuries; and to speed the healing of wounds associated with reconstructive procedures such as skin grafting and flap placement, e.g., in the repairing of wounds and aiding cosmetic procedures. In addition, the cysteine proteases can be used to treat damage to the gastric epithelium, the lung epithelium, and other internal epithelial layers.

In cases where the cysteine proteases of this invention are being used for surface wound healing, they can be administered by topical means. For topical administration, the cysteine proteases are applied directly to the site of injury as a solution, spray, gel, cream, ointment or as a dry powder. Slow release drug delivery devices directing these cysteine proteases to the injured site can be used. In addition, the cysteine proteases can be combined with topical bandages, or dressings, or sutures/staples, and with topical creams and ointments. In specific, the cysteine proteases of this invention can be used at a concentration ranging from about 50 ng/ml to about 500 ng/ml as a single application, or in dosing regimens that range from several times per day to once every few days for a period of one to several weeks. Usually, the amount of topical formulation administered is an amount which applies about 0.01 μg/cm² to about 100 μg/cm² of cysteine protease to the wound.

Complex of Proteases with Cyclodextrin

Examples of slow release drug delivery systems include β-cyclodextrin (hereinafter “βCD”). CC23a-e isoforms of the cysteine proteases can be complexed (hereinafter “P1G10”) with β-cyclodextrin by mixing the P1G10 cysteine proteases with β-cyclodextrin in ratios of 1:10, 1:20, and 1:30 w/w, respectively. The P1G10: β-cyclodextrin complex can then be incubated for 60 hours at room temperature with stirring and protected from light with aluminum foil to prevent degradation. The P1G10: β-cyclodextrin complex can be freeze dried and stored at −20 degree C., until use. Other types of β-cyclodextrins include, but are not limited to, methylated cyclodextrins, hydroxylalkylated cyclodextrins, branched cyclodextrins, alkylated cyclodextrins, acylated cyclodextrins, and anionic cyclodextrins.

The CC23a-e isoforms or cruder fractions containing these isoforms of the cysteine proteases can be prepared as follows: Dried latex (3 g) from C. candamarcensis is dissolved in 20 ml, of 1 molL⁻¹ sodium acetate solution containing 25 mmolL⁻¹ L-cysteine, 5 mmolL⁻¹ DTT and 10 mM EDTA pH 5.0. Following 30 min incubation with gentle shaking at room temperature, the solution is centrifuged in Sorvall, rotor SS-34 (10,000×g) and the supernatant filtered through gauze. The filtered solution is passed through a Sephadex G-10 column (25×400 mm) previously equilibrated with 1 mol⁻¹ sodium acetate pH 5.0. Fractions of 5 mL are collected and designed as P1G10.

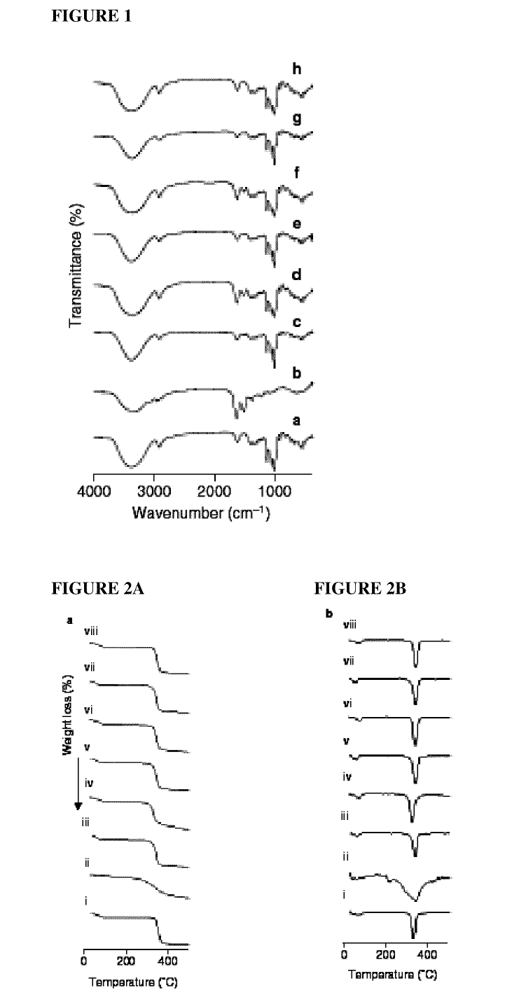

Characterization of the P1G10: βCD complex was performed using Fourier Transform Infrared (FTIR) spectroscopy, Thermogravimetry (TG), Differential Scanning Calorimetry (DSC), and X-ray Powder Diffraction (XRD) Analysis. The methods were performed as shown in the article “A Supramolecular Complex between Proteinases and [beta]-Cyclodextrin that Preserves Enzymatic Activity: Physicochemical Characterization” Biodrugs Volume 20, Number 5, 2006, pp. 283-291(9), herein incorporated by reference. As shown in FIG. 1, the FTIR spectra of P1G10: βCD complexes display a higher crystalline state than free P1G10, by comparison of the spectral frequencies below 1000 cm⁻¹. The FTIR spectra correspond to the following samples: (a) β-cyclodextrin (βCD), (b) P1G10, (c) P1G10: βCD (1:10), (d) physical mixture (PM)P1G10: βCD (1:10), (e) P1G10: βCD (1:20), (f) PMP1-G10: βCD (1:20), (g) P1G10: βCD (1:30) and (h) PMP1G10: βCD (1:30). This result indicates a reduction in lattice entropy within the complex, relative to the free protein.

### Thermogravimetric and Differential Thermogravimetric Analysis.

As shown in FIGS. 2A and 2B, the TG profile reveals an increase in thermal stability for P1G10: βCD inclusion compounds relative to free βCD, by displacement of the decomposition event in DTG curves (≈345° C.). The spectra shown correspond to TG in FIG. 2A and FIG. 2B shows the DTG analysis of following samples: (i) βCD, (ii) P1G10, (iii) P1G10: βCD (1:10), (iv) physical mixture (PM)P1G10: βCD (1:10), (v) P1G10: βCD (1:20), (vi) PMP1G10: βCD (1:20), (vii) P1G10: βCD (1:30) and (viii) PM P1G10: βCD (1:30). The sensitivity of this assay is 1.0° C. PM. In addition, a larger shifting of the breakdown temperature is observed at the highest P1G10: βCD (1:30) ratio supporting the occurrence of solid-state supramolecular non-covalent interaction between P1G10 and βCD. The TG and DTG curves represent reproducible triplicate experiments performed in a Shimadzu TGA-50 thermogravimetric analyzer, using a dynamic N₂ atmosphere ambient and a heating rate of 10° C./min and sensitivity of 1.0° C. The samples (5 mg) were placed in open pans of alumina (Al₂O₃).

### Differential Scanning Calorimetry

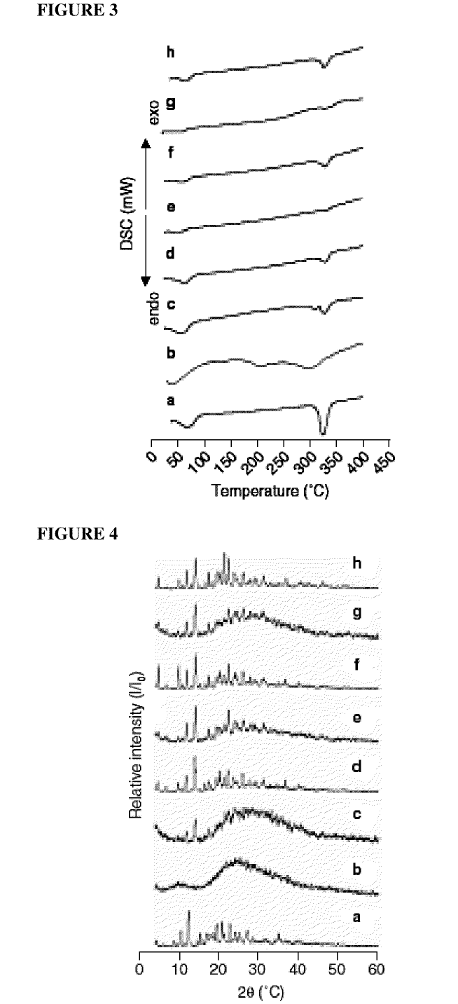

The DSC thermal profile of P1G10: βCD also changes if compared to free P1G10, βCD or the corresponding physical mixtures, as shown in FIG. 3. In FIG. 3, the samples are (a) βCD, (b) P1G10, (c) P1G10: βCD (1:10), (d) physical mixture (PM)P1G10: βCD (1:10), (e) P1G10: βCD (1:20), (f) PMP1G10: βCD (1:20), (g) P1G10: βCD (1:30) and (h) PMP1G10: βCD (1:30). The sensitivity of DSC is 0.1° C. Particularly, at high βCD ratios, there is a smoothing of the profiles at 70° C. and 350° C. The βCD DSC curves show two endothermic peaks, one at 70° C. the second at 320° C., the first attributed to a water loss and the second one, to breakdown of βCD. The P1G10 DSC curve depicted two broad endothermic peaks, one at 210° C., and the other at 300° C. The first thermal phenomenon is associated to a second order phase transition of P1G10 such as glass transition, in agreement with the profile of thermal stability observed for P1G10 on TG curves at this temperature; the second peak is linked to the protease thermal decomposition, as supported also by TG curves. The endothermic DSC signal for βCD at about 70° C. is associated to its dehydration. An overall reduction of this signal is evident when βCD is complexed to P1G10 suggesting a water exclusion process during complex formation. The βCD endothermic peaks at 70° C. and 300° C. are markedly reduced in complexes, as the βCD molar ratio increases, arguing for supramolecular interactions between the two species. The DSC profiles of 1:20 and 1:30 complexes at 70° C. and 300° C. are less pronounced than the corresponding endothermic signals for physical mixtures. These results plus the loss of signal for P1G10 at 210° C. suggest the formation of hydrogen and van der Waals interaction at high molar βCD ratios in supramolecular complexes.

### X-Ray Diffraction

FIG. 4 shows the X-Ray powder diffraction (XRD) pattern of P1G10: β-cyclodextrin (βCD). The XRD profiles of the following samples are shown: (a) βCD, (b) P1G10, (c) P1G10: βCD (1:10), (d) physical mixture (PM)P1G10: βCD (1:10), (e) P1G10: βCD (1:20), (f) PMP1G10: βCD (1:20), (g) P1G10: βCD (1:30) and (h) PMP1G10: βCD (1:30).

The freeze-drying process generally yields amorphous complexes. However, in this case a polycrystalline system is obtained as evidenced by the inclusion compound formation between P1G10 and the βCD, supported by experimental evidence. The XRD diffraction pattern of free βCD shows a polycrystalline structure similar to that described elsewhere, meanwhile the corresponding pattern of free P1G10 exhibits a highly amorphous structure. Each of the P1G10: βCD complexes had a superior amorphous character than the corresponding physical mixture. Upon complexation with βCD the P1G10 XRD pattern becomes closer to the free βCD pattern. However at 1:20 ratio this tendency reverses and the profile becomes closer to its physical mixture, suggesting a relative ordering in structure at this ratio. This notion is supported by the biological evidence showing that the amidase activity is higher and last longer at this ratio These data are in agreement with the FTIR, DTG and DSC results described earlier.

### Analysis in Solution

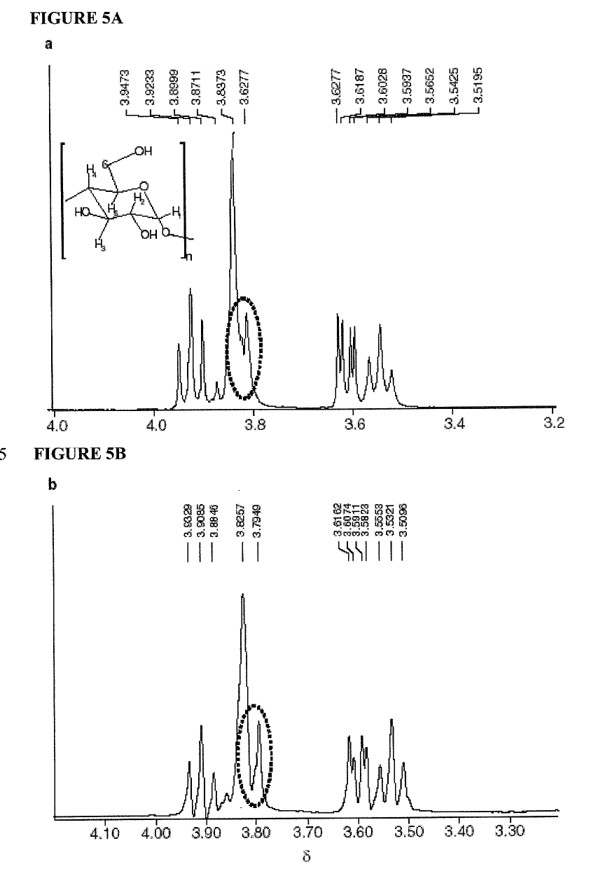

The P1G10: βCD host:guest interactions were studied by ¹H-NMR, as shown in FIGS. 5A and 5B, to investigate changes in electronic density of βCD upon complexation. ¹H-NMR spectra were obtained in triplicate assays at 298.15° K on a Brucker DRX 400 (400 MHz) spectrometer with the Brucker software package XWIN-NMR. The solutions used were 20 gL⁻¹ of pure βCD and 40 gL⁻¹ of P1G10: βCD (1:1 w/w), to assess the host:guest interaction, both dissolved in D₂O (Cambridge Isotope Laboratories, Inc—99.9% of isotopic purity). The water signal δ=4.70 was used as reference. As shown in FIG. 5, a chemical shift change and sharpening of the NMR signals in H5 and H6 region (δ≈3.5 to 3.8) occur in the P1G10: βCD system. These results are associated to disturbances of the electronic density caused by insertion of P1G10 moieties onto the βCD cavity and stabilization by non covalent interactions such as Van der Waals interactions.

### Circular Dichroism

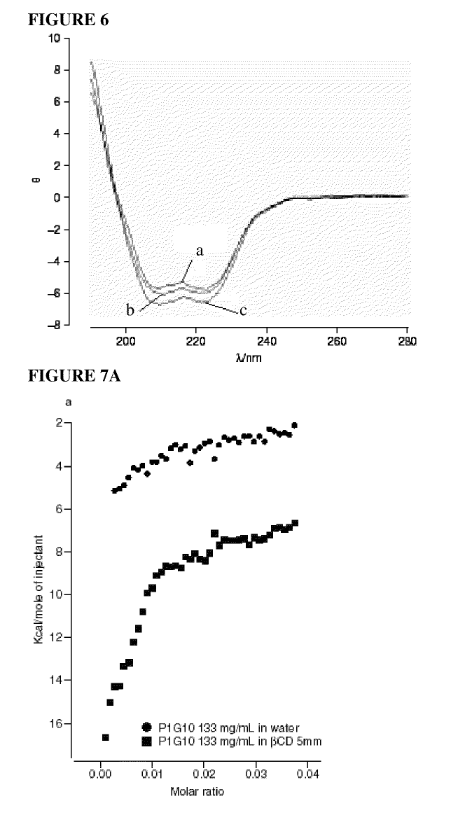

As shown in FIG. 6, the circular dichroism (CD) of P1G10: β-cyclodextrin (βCD). The CD spectra of the following samples are: (a) 0.09 g/L of P1G10, (b) 0.09 g/L of P1G10 in 0.1 mmol/L of βCD and (c) 0.09 g/L, of P1G10 in 1.0 mmol/L of βCD. The circular dichroism spectra for P1G10 fraction and P1G10: βCD system exhibited electronic transitions at 192, 209 and 222 nm featured by α-helix, attributed to π→π*(positive due to exiton coupling with π→π*perpendicular), π→π*(negative) and the red shifting of n→π*, respectively, and according with CD data for plant cysteine proteinases. These data suggest that P1G10 is primarily constituted by structures rich in α-helix, like in cysteine proteinases. The CD spectra of P1G10 and P1G10: βCD are qualitatively similar, but small changes in the P1G10: βCD spectra were observed. These changes can be explained by βCD enhancement of the α-helix character and dipolar momentum on P1G10. In addition when βCD concentration is increased from 0.1 mmolL⁻¹ to 2.0 mmolL⁻¹ (20-fold), the spectrum becomes more accentuated suggesting stronger supramolecular interactions and the induction of a stronger α-helix character.

### Microcalorimetric Measurements

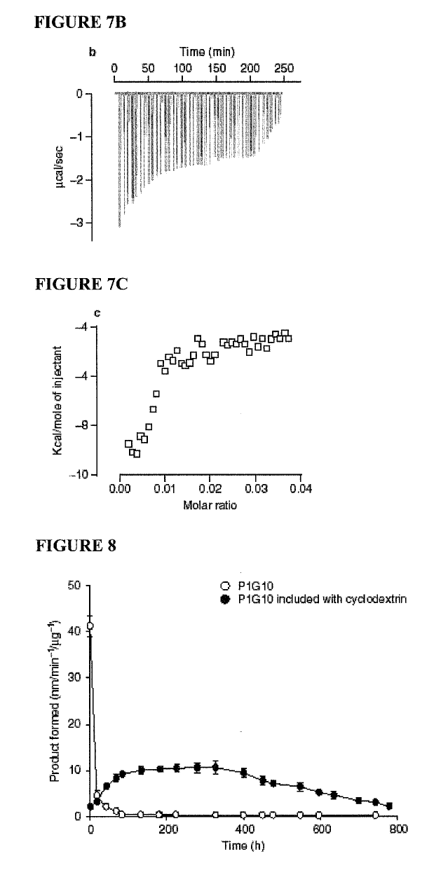

To investigate the thermodynamic parameters for the complex formation in solution, Isothermal Titrations Calorimetry (“ITC”) were conducted, as shown in FIGS. 7A, 7B, and 7C. FIG. 7A is the control curve after injection of P1G10 133 g/L in water MQ and titration curve of P1G10 133 g/L in βCD 5 mmol/L. FIG. 7B is the raw data of isothermal titration calorimetry. FIG. 7C is the adjusted titration curve after subtraction of control curve.

Calorimetric titrations were carried out in duplicate with a VP-ITC Microcalorimeter from Microcal at 298.15°K after electrical and chemical calibration. Each titration experiment consisted of 41 successive injections of aqueous P1G10 solution (133 gL⁻¹ or 1242 mmolL⁻¹ of residues, assuming an average molar weight of residues of 107 gmol⁻¹) into the reaction cell loaded with 1.5 ml, of 5 mmol-1 βCD aqueous solution, at intervals of 360 seconds. The first injection of 1 μL was discarded to eliminate the diffusion of material from the syringe into the calorimetric cell. Subsequent injections were performed at constant volume of 5 μL P1G10. The time of injection was 2 seconds. βCD concentration in the calorimeter cell varied from 5.0 to 4.3 mmolL⁻¹ and the concentration of the P1G10 from 0 to 19.0 gL-1. The raw data, as shown in FIG. 7b, were analyzed using Microcal Origin 5.0 software for ITC, after blank subtraction obtained by dilution of P1G10 in water.

A Boltzman sigmoid model (Eq. 1) was used to fit the experimental data:

$$y = \frac{A 1 - A 2}{1 + {e}^{( x - {x}_{0} ) p}} + A 2 \qquad ( 1 )$$

where y is the dependent variable described as the heat injection ΔᵢₙⱼH^{o}=dQ/d[P1G10], x is the independent variable molar ratio [P1G10]/[βCD], A1 and A2 are respectively the left and right asymptote, where A1-A2 is related to the ΔH^{o} of the process, x₀ is the inflexion point for the sigmoid fitted curve designed as stoichiometric coefficient and p is the steepness of the sigmoid curve which is related to the equilibrium constant but has no physical meaning in this treatment. By using this model the ΔH^{o}≈−4.5 Cal/mol for the P1G10: βCD supramolecular interaction was calculated, demonstrating that the complexation results in a favorable enthalpic contribution, as already described during formation of other βCD inclusion compounds. This result could be attributed to release of enthalpy rich water molecules from the βCD cavity and subsequent formation of more stable interactions by these water molecules with other water lattices. The interactions are attributed to formation of van der Waals and/or hydrogen bonding between OH moieties of βCD and P1G10 residues or to hydrophobic interactions. The stoichiometry for the system was estimated as x₀≈7.7 referent to the interaction of eight P1G10 residues per βCD molecule, in average. Thus, these data suggest the formation of a higher order complex with average stoichiometry of 1:30 (protease: βCD), in which βCD recognizes the hydrophobic moieties of the protease, creating hydrogen bonds with the external hydroxyls of βCD, generating a high order complex.

### Amidase Activity

The integrity of P1G10 complexed to βCD was measured by the amidase activity of the complex at different intervals and compared to that of free protein for one month, as shown in FIG. 8. The amidase activity of free and P1G10: βCD (1:20) complexes was determined (in quadruplicate) in the presence of 133 μmolL Pyr-Phe-Leu-pNA. Equivalent amounts of P1G10 were used in both assays. The activity data are plotted with their standard deviations (≦5%). A 2 gL⁻¹ solution of P1G10: βCD (1:20) in 0.9% saline was prepared and incubated at 37° C. for various intervals along with a solution of free βCD, as negative control. The enzyme concentration in the complex solution P1G10: βCD (1:20) was 0.09 gL⁻¹. The mass of P1G10 represents 4.7% of the total mass including βCD. In a parallel experiment free P1G10 was dissolved in saline at concentration of 0.09 gL⁻¹ and incubated at 37° C. as with βCD, using saline as negative control. Aliquots (750 μL) were withdrawn at different intervals (1-800 h) from each tube in (triplicate) and incubated in the presence of (1 μl) 50 mmolL⁻¹ Pyr-Phe-Leu-pNA in saline solution (pH 7). After 50 min incubation at 37° C. the reaction was stopped by addition of 60% (60 μL) acetic acid. The amount of p-nitroanilide released was measured spectrophotometrically at 410 nm at intervals between day 1 and 30^{th} of the assay mix incubated at 37° C. The control values obtained with free βCD or saline were subtracted from the experimental values obtained with complexed βCD or free P1G10, respectively.

As shown in FIG. 8, the free protein fraction (control) showed the highest amidase activity (100%) at initial time of 0 hour 42 nm.min⁻¹.mg protein⁻¹, but declined to 11% of the initial value by 24 h and approached zero as the incubation proceeded during the second day. In the P1G10: βCD complex assay, while minimal activity was detected by day 1 attaining about 5% of the activity displayed by free enzyme (1 h and 19 h), it rose steady and stabilized by the 2nd day at 10 nm.min⁻¹.mg protein⁻¹ and remained stable thereafter until day 16^{th}, then it gradually decreased reaching basal activity by day 29th. The activation lag observed in P1G10 βCD complex contrasts with the almost instantaneous activation of free P1G10, and must result from the diminished accessibility to proteinases when they become part of the complex. The proposed reduced accessibility to P1G10 within the complex explains the longer lifetime of the proteolytic enzymes, probably by reduction of their intrinsic rate of autolysis. The lack of initial amidase activity in the complex between 1-19 h, argues for a complete association between proteases and βCD, thus leaving no free enzyme in solution. A comparison between the amidase activity of the three complexes (1:10-1:30), indicates that complex 1:20 displays the highest amidase activity, suggesting an optimization of the interactions at this ratio (not shown).

The maximal activity attained by the P1G10: βCD (5^{th} day) represents 23% of the maximal activity exhibited by the free enzyme at 0 time. A comparative estimate of the total activity released in solution of both the free and P1G10: βCD included, reveals that the complexed enzyme remains active for a longer period, with a proteolytic potential estimated by the total area under the curve as 7.8 times superior to free enzyme. This is probably due to instability of the free enzyme preparation. The sustained activity of included P1G10 represents a desirable property for a hypothetical application of this complex since it implies a reduction in the number of applications needed to achieve the desired effect.

Thus, the complex formation leads to a prolonged stabilization of the cysteine proteases CC23a-e, represented by complex P1G10. Moreover, the total activity of P1G10: βCD does not exceed 23% of the total free proteolytic activity at any given time, limiting the adverse effects by uncontrolled proteolysis due to an excess of free P1G10, while at the same time maintain the steady state proteolysis beyond three weeks. In similar experiments conducted at pH 2 to mimic the gastric condition of an oral application (not shown), it was observed a 70% reduction of the amidase activity in the complex but keeping a measurable proteolytic effect during three weeks, as in neutral pH. Based on the ulcer protective and healing effect demonstrated for P1G10 fraction, the complexation of P1G10 with βCD improves the therapeutic efficiency of these proteolytic enzymes.

Cysteine proteases of this invention can also be used for in vitro culturing of responsive cell types, e.g., fibroblasts or epithelial cells. For such uses, the cysteine proteases can be added to the cell culture medium at a concentration of about 10 ng/ml to about 100 ng/ml. In addition, cells grown under growth factor stimulation can be used as a source of expanded cell populations for grafting purposes. For all of these applications, the cysteine proteases of this invention may be used alone or in combination with other proliferative factors, debriding agents and biologically active agents. Other debriding agents include trypsin, collagenase dextranomer, cadexomer iodine, and hydrogels, e.g., INTRASITE GEL®. (Smith & Nephew Healthcare Ltd), STERIGEL®. (Seton Healthcare Group plc) and GRANUGEL®. (CovaTec UK, Ltd.).

### Example 1

### Purification of CC23a-e from Latex Extract of Carica Candarmacensis

Crude latex extract from Carica candarmacensis was dissolved in 1 M sodium acetate buffer, 1 mM EDTA, 0.01% sodium azide, pH 5.0. This was filtered through 0.22 μm filters and the protein content was determined by absorption at 280 nm using an A_{1%,280} of 20.1 (Murachi, T. and Yasui, M., Biochemistry, 4, 2275-2282, 1965). The preparation was then chromatographed on a G-10 filtration column (Amersham-Pharmacia) and eluted with the same buffer. The first peak corresponded to the bulk protease activity and was pooled and applied onto a CM-Sephadex column (Amersham-Pharmacia). The protein fractions were eluted with a linear gradient of 0.05M to 1.0M sodium acetate, pH 5.0 (essentially as described previously for chymopapain in Buttle, D. J., and Barrett, A. J., Biochem. J., 223, 81-88, (1984)). The second protein peak containing the protease activity termed CC23a-e was pooled and applied onto a Mono S HR 10/10 column of a FPLC system (Pharmacia). The protein mix was eluted with a gradient of 0.002M to 1.0M NaCl at pH 9.2.

Plots of A₂₈₀ and gradient composition were provided automatically. The fractions were stored, tightly capped at 4° C. until assayed and further processed. The five peaks eluting between 0.02M and 0.39M NaCl were taken and designated as peaks CC23a-e. Each of the peaks CC23a-e were concentrated and dialyzed in an Amicon PM10 concentration chamber against 10 mM sodium acetate, 1 mM EDTA, 0.01% sodium azide, pH 6.5 at 4° C.

### Example 2

### Proliferation Assays

The above purified proteins, CC23a-e, their metabolites, or the crude extracts from which were derived, were assayed for their proliferative activity as follows. The mitogenic fractions were assayed as described below, using either fibroblasts (i.e., L929), epithelial cells (i.e., human keratinocytes), or human mammary cells (i.e., MDA MB 231). Fractions (5-50 ng/ml) were tested for proliferative activity by measuring the effect of aliquots of the fractions on DNA synthesis. This was accomplished by actually measuring the proliferation of L929, MDA MB 231 and keratinocytes cells by monitoring the incorporation of [³H]-thymidine into DNA and/or by measuring the increase in cell number, using MTT as an indicator.

Cells were plated at 1−2×10⁴ cells/well (Costar, Cambridge, Mass.) in RPMI 1640 (GIBCO, Grand Island, N.Y.) supplemented with 10% fetal calf serum (GIBCO, Grand Island, N.Y.) and antibiotics (100 units/ml penicillin and 100 μg/ml streptomycin sulfate, GIBCO, Grand Island, N.Y.). After 24-48 h at 37° C., 2, 5% CO₂, cells were washed twice with phosphate-buffered saline, and the medium was replaced with 0.5% FBS RPMI 1640 supplemented with antibiotics for 24-36 h before the assay.

Test samples were then added at the specified concentrations during 24 to 72 h. One μCi [³H]-thymidine/well (Amersham Pharmacia) was added and remained in the assay for 18 h before arresting the reaction. The reaction was stopped by 2 washes with RPMI, 2 washes with 5% TCA and 2 washes with 95% ethanol. The remaining radioactivity was measured following treatment with Aquasol (Amersham-Phamacia) in a scintillation counter. The following table, Table 1, summarizes the proliferative effect of CC23a-e fractions on L929 cells.

TABLE 1

| Protein tested | 10 ng/mL | 20 ng/mL | 50 ng/mL |
|---|---|---|---|
| Crude Fraction | 105% | 17% | 31% |
| CC23a (SEQ ID NO: 1) | 69% | 61% | 98% |
| CC23b (SEQ ID NO: 2) | 54% | 93% | 67% |
| CC23c (SEQ ID NO: 3) | 18% | 11% | 16% |
| CC23d | 8% | — | 18% |
| CC23e | 61% | 36% | 32% |
| Papain | — | 25% | — |

The determined proliferative activity (assayed as [³H]-thymidine incorporation into L929 cells) showed that the purified form of CC23a-e was not destroyed by heating to 90° C. for 5 minutes but was significantly destroyed (60%) by exposure to the cysteine protease inhibitor E64 for a period of 30 min (data not shown).

As shown in Table 1, the purified protein CC23a stimulated close to 100% proliferation of L929 fibroblast cells at a concentration of 50 ng/ml, while the maximal stimulatory effect of CC23d was 18% at 50 ng/ml. Papain from Sigma, but not chymopapain, showed 25% stimulatory affect at 20 ng/ml using the same protocol. The proliferative effect of each protease tested was also reduced by the addition of the inhibitor E64.

### Example 3

### N-Terminal Sequencing

The amino acid sequence of the purified form of the protease CC23a, CC23b, and CC23c was determined. Approximately 1.7 μg of protein, obtained after cation exchange-MonoS chromatography, was loaded onto an Applied Biosystems gas-phase protein sequencer. Two hundred and fourteen rounds or fifteen rounds of Edman degradation were carried out, and identification of amino acid derivatives was made with an automated on-line PTH-amino acid analyzer (model 477A, Applied Biosystems, Foster City, Calif.).

The two hundred and fourteen rounds of Edman degradation of CC23a resulted in the identification of the 214 amino acid residues, depicted as SEQ ID NO: 1 (YPESIDWRQKGAVTPVKDQNPCGSCWAFSTVATVEGINKIVTGKLISLSEQELLDCDRR SHGCKGGYQTTSLQYVVDNGVHTEKVYPYEKKQGKCRAKDKQGPWVKIITGYKRVPSN DEISLIKAIATQPVSVLVESKGRAFQFYKGGVFGGPCGTKLDHAVTAVGYGKDYILIKNS WGLRWGDKGYIKIKNASGNSEGICGVYKSSYFPIKGYQ).

The two hundred and fourteen rounds of Edman degradation of CC23b resulted in the identification of the 214 amino acid residues, depicted as SEQ ID NO:2 (YPGSVDWRQKGAVTPVGDQNPCGSCWAFSTVATVEGINKIVTGHLISLSEQELLDCDR RSHGCKGGYQTGSLQYVVDYGVHTEYVYPYEKKQGKCRAKDKQGPKVQITGYKRVPT NDEISLIKVIANQPVSKLIESKGRSFHFYRGGIYKGPCGTRLDHAVTAIGYGKDYILIKNS WGPNWGEKGYIKIKNASGKSEGICGVYKSSYFPTKEYQ).

### Example 4

### Treatment of Wound of Patient

A patient diagnosed with a non-healing wound, specifically a lesion on the skin unable to heal without intervention, is selected for treatment with the cysteine protease CC23a. The purified protease CC23a can be used at a concentration of about 200 ng/ml as a single application, twice daily for a period of about a week. The amount of topical formulation administered to a patient is an amount which applies about 25 μg/cm² of CC23a to the lesion.

After treatment for approximately a week, the non-healing lesion shows signs of undergoing healing. Alternatively, the purified protease CC23b can be used at a similar concentration as a single application, twice daily for a period

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pharmaceutical composition comprising a first protein having an amino acid sequence comprising SEQ ID NO: 1 and a second protein having an amino acid sequence comprising SEQ ID NO: 2, wherein the composition further comprises a pharmaceutically acceptable carrier.

2. The pharmaceutical composition of claim 1, wherein the pharmaceutical composition comprises the second protein having an amino acid sequence comprising SEQ ID NO: 2.

3. The pharmaceutical composition of claim 1, further comprising a plurality of proteins having a protease activity of CC23d and CC23e.

4. The pharmaceutical composition of claim 1, further comprising a slow release delivery system, wherein the slow release delivery system comprises cyclodextrin.

5. The pharmaceutical composition of claim 2, further comprising a slow release delivery system, wherein the slow release delivery system comprises cyclodextrin.

6. The pharmaceutical composition of claim 1, wherein the first protein and the second protein are in a concentration ranging from about 0.01 μg/cm² to about 100 μg/cm².

7. The pharmaceutical composition of claim 2, wherein the second protein is in a concentration ranging from about 0.01 μg/cm² to about 100 μg/cm².

8. A method of treating wounds comprising administering a pharmaceutical composition including a first protein having an amino acid sequence of SEQ ID NO: 1 and a second protein having an amino acid sequence of SEQ ID NO: 2.

9. The method of treating wounds of claim 8, wherein the pharmaceutical composition comprises the second protein having an amino acid sequence of SEQ ID NO: 2.

10. The method of treating wounds of claim 8, wherein the first protein and the second protein comprise a concentration ranging from about 50 ng/ml to about 500 ng/ml in a single application.

11. The method of treating wounds of claim 9, wherein the second protein comprises a concentration ranging from about 50 ng/ml to about 500 ng/ml in a single application.

12. The method of treating wounds of claim 8, wherein the pharmaceutical composition comprises a topical application.

13. The method of treating wounds of claim 12, wherein the topical application is selected from a group consisting essentially of a solution, a spray, a gel, cream, an ointment, or a dry powder.

14. The method of treating wounds of claim 12, wherein the wounds include acute wounds such as burns, incisions and injuries.

15. The method of treating wounds of claim 12, wherein the wounds include chronic non-healing wounds such as fall-thickness dermal ulcers, pressure sores, venous ulcers, and diabetic ulcers.

16. The method of treating wounds of claim 12, wherein the wounds are associated with reconstructive procedures.

17. The method of treating wounds of claim 12, wherein the wounds are associated with the gastric epithelium, lung epithelium or other internal epithelial layers.

18. A method for stimulating the mitogenic activity of in vitro cultured cells using a pharmaceutical composition comprising a first protein having an amino acid sequence of SEQ ID NO: 1 and a second protein having an amino acid sequence of SEQ ID NO: 2.

19. The method of claim 18, wherein the pharmaceutical composition comprises the second protein having an amino acid sequence of SEQ ID NO: 2.

20. The method of claim 18, wherein the first protein and the second protein comprises a concentration ranging from about 50 ng/ml to about 500 ng/ml in a single application.
